(19) United States
(12) Patent Application Publication
(10) Pub. No.: US 2012/0282676 A1
(43) Pub. Date: Nov. 8, 2012

(54) Compositions and Methods for Enhancing Lipid Production in Marine Microalgae

(75) Inventors: Paul Falkowski, Princeton, NJ (US); Miguel Frada, Porto (PT); Kevin Wyman, East Brunswick, NJ (US); James Gibson, Basking Bridge, NJ (US)

(73) Assignee: Old Queens, New Brunswick, NJ (US)

(21) Appl. No.: 13/455,746

(22) Filed: Apr. 25, 2012

Related U.S. Application Data
(60) Provisional application No. 61/478,910, filed on Apr. 25, 2011.

(51) Int. Cl. C12N 1/12

(52) U.S. Cl. 435/257.1

(57) ABSTRACT

Methods for enhancing lipid production and growth rate of marine algae are provided.

This application claims priority to U.S. Provisional Application No. 61/478,910 filed Apr. 25, 2011, the entire disclosure being incorporated by reference herein as though set forth in full.

### FIELD OF THE INVENTION

This invention relates to the fields of lipid metabolism and biofuel production. More specifically, the invention provides algal cells and methods for culturing the same which enhance endogenous lipid levels and growth rates thereby facilitating production of biodiesel fuels.

### BACKGROUND OF THE INVENTION

Numerous publications and patent documents, including both published applications and issued patents, are cited throughout the specification in order to describe the state of the art to which this invention pertains. Each of these citations is incorporated herein by reference as though set forth in full.

Eukaryotic microalgae are extremely diverse, thriving in all aquatic ecosystems, where they constitute the base of the food chain and are the major drivers of elemental cycling. The ability to proliferate over such a wide range of environments is largely reflected by their physiological resilience facing environmental fluctuations. This includes the ability to adjust their lipid metabolism under stress conditions (e.g. nutrient limitations, temperature and salinity variations), altering membrane fluidity and the volume of lipidic reserves (e.g Thompson 1989, Murata and Los 1997, Gurschina and Harwood 2006, Hu et al. 2008). Of all nutrient-related stresses evaluated, nitrogen deprivation is the single most critical factor affecting lipid metabolism in algae. A general trend towards a 1.5-2 fold increase of fatty acids as a response to nitrogen deficiency has been observed in numerous strains (Hu et al. 2008). Nitrogen deprivation limits amino acid production and protein synthesis decreases (Falkoswki et al. 1989, Berges et al. 1996). Growth and photosynthesis are therefore highly impaired, leading to a concomitant accumulation of lipids (namely triacylglycerides, TAGs) used as carbon and energy provisions (Hu et al. 2008). The ability of microalgae to modulate and augment the lipid quotas under particular circumstances has stimulated its exploitation as a source of oils for biofuel and biomaterials (Chisti 2007, Dismukes et al. 2008, Hu et al. 2008). However, the lipid yields obtained from algal mass culture efforts fall short of the theoretical maximum and make the algal oil technology prohibitively expensive (Sheehan et al. 1998, Hu et al. 2006). A deeper understanding of the growth conditions and of the physiological and genetic factors regulating lipid synthesis as well as the implementation of novel strategies to induce lipid accumulation in microalgae are therefore required to manipulate these organisms and consequently maximize oil yields.

### SUMMARY OF THE INVENTION

In accordance with the present invention, a method for increasing lipid production and growth rate in marine alga is provided. An exemplary method entails culturing microalgae in the presence of NH₄⁺ as the sole nitrogen source and exposing the culture to an effective amount of tungsten, the tungsten causing inactivation of nitrate reductase, the combination of ammonium and tungsten exposure resulting in elevated fatty acid accumulation and enhanced growth rate when compared to microalgal cells grown in NO₃-replete and nitrogen free medium in the absence of tungsten and NH₄⁺. In a preferred embodiment the resulting lipids are harvested for the production of biofuel.

The method may further comprise maintaining the microalgae under high light conditions of approximately, 950 μE which also enhances lipid production therefrom.

### BRIEF DESCRIPTION OF THE DRAWINGS

FIG. 1. Daily rates of fatty acid accumulation per Phaeodactylum tricornutum cell and per culture volume. 1—NO₃+Mo; 2—NO₃−Mo; 3—NO₃−Mo+W; 4—N-free medium; 5—NH₄−Mo; 6—NH₄−Mo+W.

FIG. 2. Nitrate reductase (NR) western-blot profile. The arrow indicates the position of NR protein. Mk, molecular ladder (represented 100 and 75 kbp); 1—NO₃+Mo; 2—NO₃−Mo; 3—NO₃−Mo+W; 4—NO₃— free medium; 5—NH₄−Mo; 6—NH₄−Mo+W.

FIG. 3. Carbon metabolites of Phaeodactylum tricornutum under the experimental conditions: 1—NO₃+Mo; 2—NO₃−Mo; 3—NO₃−Mo+W; 4—N-free medium; 5—NH₄−Mo; 6—NH₄−Mo+W. Abbreviations: MaCoA, malonyl CoA; AcCoA, acetyl CoA; MAL, malate; SUC, succinate; AKG, alfa-ketoglutarate; GLU, glutamate; GLN, glutamine.

FIG. 4. Merged epifluorescence microscope images of Phaeodactylum tricornutum cells stained with BODIPY (for membrane lipid and lipid bodies; green fluorescence) and chloroplasts autofluorescence (red). A—NO₃+Mo; B—N-free medium; C—NO₃−Mo+W; D—NH₄−Mo. Scale=5 μm.

FIG. 5. Relationship between growth rate (d⁻¹) and fatty acid per cell (pg). The groups correspond to: a—NO₃+Mo and NO₃−Mo; b—NO₃−Mo+W and N-free; c—NH₄−Mo and NH₄−Mo+W. The black line represents the trendline among all conditions (equation 1: y=−1.97x+5.03, R²=0.41), whereas the grey line represents only the trendline between a and b conditions (equation 2: y=−3.09x+5.46, R²=0.99).

FIG. 6. Liquid-chromatography mass-spectrometry (LC-MS) plot representing the relative content of triacylglycerides (TAG) species between cells growing with nitrate or ammonium as nitrogen sources. Total lipids from P. tricronutum cells growing with nitrate or ammonium as nitrogen source (from the experiments described in the main text) were extracted with the Bligh-Dyer method (Bligh and Dyer) and analysed by liquid-chromatography mass-spectrometry (LC-MS).

FIG. 7. Comparison of growth, fatty acid content and rates of fatty acid accumulation per volume of culture of the green microalgae Dunaliella terciolecta with nitrate or ammonium as nitrogen source and supplemented with tungsten.

FIG. 8. Flow cytometric comparison of cell stained with the lipophilic dye BODIPY of Phaeodactylum tricornutum cells growing under different nitrogen regimes

### DETAILED DESCRIPTION OF THE INVENTION

Nitrogen limitation is the single most critical factor affecting lipid metabolism in microalgae, leading to significant increases of the cellular lipid content. In this work we blocked NO₃− assimilation as a direct strategy to induce nitrogen starvation and potentiate lipid accumulation in the marine diatom Phaeodactylum tricornutum. For that we inactivated the nitrate reductase (NR) enzyme using sodium-tungsten (W). We then compared the effects of W upon fatty acid and metabolite pool sizes and the physiological efficiencies for growth and fat production relative to cells growing in NO₃-replete and nitrogen-free media. We included supplementary conditions using NH₄⁺ as nitrogen source with and without W to control for secondary effects of W. In general, the addition of W to NO₃− grown cells resulted in high levels of lipid accumulation, similarly to cells in nitrogen-free medium. The specific regulation of NR is therefore a mechanism to augment lipid accumulation. However unexpectedly from all conditions, NH₄⁺ medium promoted the highest degree of fatty acid accumulation per cell, which seems to be related to increased flux of carbon towards lipid biosynthesis, probably as a mechanism of photoprotection. Interestingly the addition of W to cells growing with NH₄⁺ boosted cellular growth rates, which led to over 30% higher rates of fat accumulation per unit volume per day. Finally we demonstrated the same NH₄⁺ and W effects in another microalgae, Dunaliella terciolecta. From a biodiesel perspective these results indicate that NH₄⁺, if suitable to a given microalgal, should be the preferred nitrogen-source. Furthermore the mechanisms by which W enhances growth should be investigated in order to genetically-engineer microalgae and potentiate rates of oil production.

The following materials and methods are provided to facilitate the practice of the present invention.

### Experimental Conditions.

Axenic cultures of the raphid pennate diatom P. tricornutum Bohlin accession Pt1 8.6 (deposited as CCMP2561 in the Provasoli-Guillard National Center for Culture of Marine Phytoplankton, de Martino et al. 2007 and Bowler et al. 2008) were acclimatized for 2 weeks to 3 media formulations based on sterile artificial seawater (Berges et al. 2001) amended with f/2 (Guillard 1975) concentrations of NO₃− or NH₄+ (added as NH₄Cl), phosphate, silicate, vitamins and trace metals (with or without molybdenum, Mo): 1—NO₃⁻ medium with Mo (NO₃+Mo); 2—NO₃⁻ medium without Mo (NO₃−Mo); 3—NH₄⁺ without Mo (NH₄−Mo). The removal of Mo was imperative to obtain complete inactivation of NR with sodium tungsten (W) in subsequent tests. This was also the reason to perform all experiments in artificial medium. The inclusion of a condition with NH₄⁺ as nitrogen source was established in a first instance to control for secondary effect of sodium tungsten upon the cells.

During the period of acclimation the cultures were maintained at 18° C., under continuous light using liner cool fluorescent lamps emitting white light at 300 μmol quanta·m⁻²·s⁻¹, and 0.2 μm filtered air bubbling. Periodic dilutions with fresh media were also executed to main the cells in exponential phase of growth.

To initiate the experiment, exponentially growing cells where pelleted by centrifugation (8000 rpm, 18° C., 10 min). Each cell pellets was subdivided into two different base media, including the acclimation media, at an initial concentration of 3×10⁵ cells·ml⁻¹ (in 1 L of volume) constituting 6 final conditions as schematized in Table I. The light intensity and temperature were maintained as described above. After 3 days (72 h) of growth samples were collected and processed for further analyses (described bellow). Experiments were performed at least in triplicate. The cellular densities were monitored with a Coulter counter multisizer 3 (Beckman Coulter Inc, Fullerton, Calif., USA).

TABLE I
Schematization of media conditions

| Acclimation | Experiment |
|---|---|
| NO₃ + Mo | NO₃ + Mo |
| | Medium without nitrogen source (N-free medium) |
| NO₃ − Mo | NO₃ − Mo |
| | NO₃ − Mo, plus 0.9 mM sodium-tungsten (NO₃ − Mo + W) |
| NH₄ − Mo | NH₄ − Mo |
| | NH₄ − Mo, plus 0.9 mM sodium-tungsten |
| | (NH₄ − Mo + W) |

### Photophysiology and Intracellular Carbon and Nitrogen Pools.

The maximum photochemical quantum yield of PSII (variable fluorescence/maximum fluorescence ratio [Fv/Fm]) of the cultures was measured as an indication of photosynthetic health using the fluorescence induction and relaxation system (FIRe; Satlantic Instrument, Gorbunov 2005). Chlorophyll was extracted with 90% acetone from cells collected (10-20 ml of culture) by vacuum filtration onto Whatman 25 mm GF/F filters. The absorption spectrum was determined spectrophotometrically (375-750 nm scan) using an SLM-Aminco DW-2000 spectrophotometer and the chlorophyll concentrations calculated with the equations of Jeffrey and Humphrey 1975. Cells for carbon and nitrogen contents were harvested (10-20 ml) by vacuum filtration onto pre-combusted Whatman 13 mm GF/F filters and analyzed on a CHN analyzer (Na 1500 series 2, Carlo Erba Instruments). In vivo absorption spectra was measured with an SLM-Aminco DW-2000 spectrophotometer, using fresh unconcentrated cell suspensions. The values obtained were used to calculate the wavelength-specific cross-section (K_{cλ} here referred as a*) of optical absorption normalized to chlorophyll a (Falkowski et al. 1985). The quanta requirement for gross efficiency of transduction of light energy at a given irradiance intensity to chemical energy (total carbon or for fatty acid accumulation) was calculated with a simplified version of the model proposed by Falkowski et al. 1985:

1/λ=(a*×(Chla/C)×I_{μ}×1040)/μ(mol quanta·mol C⁻¹)

Where φ represents the quantum yield for growth or fatty acid accumulation (mol carbon). 1/φ is referred to as quantum requirement. A* the absorption cross-section (m2·mg⁻¹ Chlorophyll a), Chl a and C the biomass (mg) of chlorophyll a and total carbon (this variable was replaced by the biomass of fatty acids or carbohydrates to calculate the respective yields), I_{μ} the irradiance (μmol quanta·m⁻²·s⁻¹) and 1040 constant used to convert units from μmol quanta m⁻² s⁻¹ to mol quanta m⁻² d⁻¹ and from mg C to mol C. μ is the specific growth rate (d⁻¹).

### Nitrate Reductase Activity Assay.

Cellular NR activity assays were based on a method described in Berges and Harrison, 1995. Briefly, 100 ml of cultures (after 72 h exposed to each treatment) were gently collected by vacuum filtration on GF/F filters, plunged into extraction buffer (200 mM phosphate buffer pH7.8; 1 mM dithiothreitol; 0.3% polyvinylpyrrolidone; 3% bovine serum albumin; 0.1% triton; 5 mM ethylenediaminetetraacetic acid) at 4° C. and ground for 30 sec with a Teflon pestle. Homogenates were spun down and 200 μl of the supernatant was incubate at 20° C. with 200 KNO₃, 780 μl 200 mM phosphate buffer pH7.8. Reactions were stopped with 2 ml 550 mM zinc acetate at time 0 (blanks) and after 30 min. Debris where remove by centrifugation and the supernatant was color developed with 100 μl of 1:1 sulfanilamide N-(1-naphthyl)-ethylenediamine; 2M HCl solution. Absorbances were read with an Agilent 8453 spectrophotometer (Agilent Technologies, Santa Clara, Calif., USA) at 543 nm against the blanks. A standard curve prepared with a gradient of NO₂⁻ concentrations (0 to 0.1 μmol·ml⁻¹) was processed as described above and used to calculate the NR activity per cell (μmol NO₂⁻·min⁻¹·cell⁻¹).

### Fatty Acids Extraction and Analysis.

Fatty acid methyl esters (FAME) extraction and methylation was based on a method developed by Rodriguez-Ruiz et al., 1998. Brielfy, 5×10⁷ cells were collected onto Whatman GF/F filters and were inserted into a glass vial with 2 ml of 1:20 acetyl chloride:methanol mixture, 1 ml of hexane and 25 μg of heptadecanoic acid (Sigma-Aldrich) used as internal standard for methyl ester quantification. The vials were sealed with a Teflon-lined cap and boiled in a water-bath for 1 h. After incubation the vials were cooled to room temperature and 1 ml of milliQ water was added to each sample. Samples were vortexed and centrifuged (5 min, 3000 rpm) to allow phase separation and the top hexanic phases were transferred with glass-pipettes to GC vials with Teflon-lined caps (Fisher Scientific). FAME analyses were performed in a gas-chromatographer (GC 2010 Shimadzu, Japan) equipped with a TR-FAME (0.25 μm×60 m) column (Thermo electron corporation, USA) and a flame ionization detector (FID). Helium was used as the carrier gas at 26 psi. The injector and detector temperatures were 240° C. and 250° C. respectively. The time-oven temperature program was: 140° C. (5 min) increasing up to 240° C. at 4° C.·min⁻¹. FAMEs were identified by reference to authentic standards (Supelco™ 37 component FAME, Sigma-Aldrich) processed and analyzed under the same conditions.

### Protein Quantification and NR Western-Blot.

Total proteins were extracted from 1×10⁸ cells collected in polycarbonate membranes. Cells were resuspended in 300 μl of 4% SDS, 100 mM Na₂CO₃ and 15 μl of protease inhibitor cocktail solution (Sigma-Aldrich), sonicated on ice (3×15 sec., Microson set at power 2) and centrifuged (16000 g, 5 min). Protein concentrations were determined with the bicinchoninic acid method (BCA, Pierce). For western-blotting, 30 μl of 1M DTT and 300 μl of a solution containing of 4% SDS, 15% glycerol and 0.05% bromothymol blue were added to each sample and boiled for 5 min. 12 μg of protein from each sample were loaded onto a pre-cast 4-20% Tris-HCl gel (BIORAD), ran for 1 h (100 mV) and transferred electrophoretically to a PVDF membrane. Blots were probed for 1 h with anti-NR assimilatory (1:500) purchased from Agrisera (part no. AS08-310). Subsequently, an HRP-conjugated polyclonal goat anti-rabbit IgC antibody (Pierce, 1:10000) was used followed by chemifluorescence detection (Amersham™ ECL Plus, RPN2132). Image acquisition was performed with a molecular imager XR+ system (BIORAD, USA).

### Metabolite Profiling.

Metabolite profiles were obtained via the method described by Bennette et al. (2010). Cells were quenched via rapid filtration of 14.3 million cells followed by immediate transfer of filters to 1.8 mL of 80:20 methanol:water at −20° C. in 35×10 mm Petri dishes. Filters were incubated at −20° C. for 15 minutes and scraped clean of cells in the extraction solvent. The 1.8 mL of extraction solvent with cells was transferred to 2 mL eppendorf tubes, followed by a 0.25 mL 80:20 methanol:water at −20° C. wash of the filter to collect remaining quenched cell material. The eppendorf tubes were spun at 4° C., and supernatant was collected. To the cell pellet, 100 μL 80:20 methanol:water at −20° C. was added, vortexed, incubated at −20° C. for 15 minutes, and pelletted. The 100 μL was combined with the original supernatant, and 50 μL transferred to GC vials. The analysis of the metabolites was achieved in one 35-minute LC-MS/MS run on a 1200-series LC with a 6410 QQQ MS employing reversed-phase ion pairing chromatography (Agilent Technologies, Santa Clara, Calif., USA). Standards for each metabolite, run with sample background, were used for quantification. The column used was a Synergi 2.5μ hydro RP 100A, size 100×2 mm (Phenomonex, Torrance, Calif., USA).

### Lipid Body Imaging.

Intracellular lipid bodies were stained with 10 μg·ml⁻¹ BODIPY (4,4-difluoro-3a, 4a-diaza-s-indacene) probe (D-3922, Molecular Probes, Invitrogen, USA). Cells were subsequently observed with an inverted epifluorescence microscope Olympus IX71 (Olympus, Japan) equipped with a mercury X-CITE 120 lamp (EXFO, Canada), using the 482/536 nm excitation/emission filter. Images were acquired with a Qlmaging Retiga Exi SVGA high-speed monochromatic cooled CCD camera system and IPLab for Mac (v3.9) for image processing and analysis.

Effect of Nitrogen Sources and Tungsten on the Marine Green Algae Dunaliella terciolecta.

Non-axenic cultures of the chlorophyte Dunaliella terciolecta CCMP1320 were maintained prior experimentation in sterile artificial seawater (Berges et al. 2001) amended with f/2 (Guillard 1975) (with molybdenum) at 18° C., continuous 300 μmol quanta·m⁻²·s⁻¹ light irradiance and air bubbling. To test the effect of nitrate sources and the effect of tungsten on the accumulation of cellular fatty acids 2 medium variations were initially performed aiming to produce acclimatized biomass for later tests: 1) medium with molybdenum (NO₃⁻+Mo) as described above; 2) medium+Mo and 0.9 mM NH₄⁺ as nitrogen source (NH₄⁺+Mo). Under these conditions cells where grown up to late-exponential phase, harvested by centrifugation (8000 rpm, 18° C., 10 min). NO₃⁻+Mo cell pellets were used to inoculate fresh NO₃⁻+Mo medium and the NH₄⁺+Mo cell pellets were divided into new fresh NH₄⁺+Mo and one with 0.9 mM of tungsten (NH₄⁺+Mo+0.9 mMW). Experiments were performed in 1 L of medium, initiated with 3×10⁵ cells·ml⁻¹ and harvested at the end of 3 days of growth. Cellular densities and average cell sizes were daily monitored using a Coulter counter (Beckman Coulter multisizer 3). Epifluorescence microscopy images and fatty acid content analysis were both performed as described above.

Flow cytometry.

P. tricornutum Bohlin accession Pt1 8.6 (CCMP256) lipid (from all treatment described in this work) were stained with BODIPY probe (4,4-difluoro-3a,4a-diaza-s-indacene, D-3922, Molecular Probes, Invitrogen) as described in the microscopy section and incubated in the dark for 30 min. 2 μm fluorescent beads (fluorebrite calibration grade size kit Polysciences, Inc.) were included in the samples and used as internal reference. Subsequently samples were analyzed with an InFlux Model 209S Mariner flow cytometer (BD biosciences, San Jose Calif.). Stained cells were excited at 488 nm and detected at 520 nm (BODIPY) and 640 nm (Chlorophyll auto-fluorescence). Data was acquired in logarithmic scale. Unstained cells were also analyzed and used as normalization blanks for each treatment. Data was analyzed with the flowjo software version 7.6 (Tree Star, Inc.).

Statistical analyses.

The Student's t-test was used to compare the physiological effect of the treatments relative to the control condition (NO₃+Mo).

The following example is provided to illustrate certain embodiments of the invention. It is not intended to limit the invention in any way.

### Example I

### The Effects of Nitrate Reductase Inactivation with Tungsten and of Ammonium as a Source of Nitrogen on Fatty Acid Accumulation in the Marine Diatom Phaeodactylum tricornutum

We compared several physiological and biochemical characteristics of P. tricornutum cells after 3 days growing in different nitrogen regimes, i.e. having NO₃− or NH₄+ as nitrogen sources and under nitrogen starvation, using nitrogen-free medium (N-free) or by inactivating NO3− reduction with W (Table IIA).

### Comparative Physiology

TABLE IIA
General cellular, physiological, and biochemical characteristics of P. tricornutum cells. Values
represent mean (± standard deviation, n ≧ 3).

| | Experimental conditions | | | | | |
|---|---|---|---|---|---|---|
| | NO₃ + Mo | NO₃ − Mo | NO₃ − Mo + W | N-free | NH₄ − Mo | NH₄ − Mo + W |
| μ (day⁻¹) | 0.97 ± 0.10 | 0.93 ± 0.10 | 0.47 ± 0.02 | 0.41 ± 0.04 | 0.87 ± 0.05 | 1.02 ± 0.03 |
| Fv/Fm | 0.61 ± 0.01 | 0.61 ± 0.04 | 0.50 ± 0.00 | 0.26 ± 0.00 | 0.58 ± 0.03 | 0.58 ± 0.02 |
| C cell⁻¹ (pg) | 12.4 ± 5.46 | 17.3 ± 2.52 | 20.7 ± 0.17 | 9.37 ± 0.28 | 26.7 ± 5.37 | 13.7 ± 1.12 |
| N cell⁻¹ (pg) | 1.97 ± 0.71 | 2.79 ± 0.42 | 3.08 ± 0.06 | 0.64 ± 0.02 | 4.03 ± 0.85 | 1.83 ± 0.35 |
| C:N (mol mol⁻¹) | 7.22 ± 0.51 | 7.28 ± 0.72 | 7.86 ± 0.10 | 17.1 ± 0.69 | 7.76 ± 0.17 | 9.28 ± 0.34 |
| Chl cell⁻¹ (pg) | 0.21 ± 0.02 | 0.15 ± 0.04 | 0.09 ± 0.01 | 0.04 ± 0.00 | 0.46 ± 0.07 | 0.20 ± 0.11 |
| a* (m² mg⁻¹ Chl a) | 0.02 ± 0.00 | 0.02 ± 0.01 | 0.04 ± 0.01 | 0.09 ± 0.01 | 0.01 ± 0.00 | 0.02 ± 0.01 |
| 1/Ø (quanta C⁻¹) | 142 ± 55.7 | 95.4 ± 33.2 | 381 ± 39.0 | 1340 ± 271 | 99.9 ± 12.3 | 84.2 ± 20.7 |
| Fatty acids cell⁻¹ (pg)^{a} | 2.40 ± 0.09 | 2.66 ± 0.25 | 3.99 ± 0.24 | 4.21 ± 0.33 | 4.33 ± 0.60 | 3.38 ± 0.16 |
| Fatty acid: C (pg pg⁻¹) | 0.23 ± 0.10 | 0.17 ± 0.01 | 0.19 ± 0.01 | 0.45 ± 0.02 | 0.16 ± 0.01 | 0.25 ± 0.03 |
| 1/Ø (quanta fa⁻¹) × 10 | 643 ± 66.4 | 648 ± 252 | 1989 ± 274 | 3009 ± 728 | 612 ± 66.5 | 369 ± 125 |
| Protein cell⁻¹ (pg) | 5.38 ± 0.07 | 5.51 ± 0.71 | 5.32 ± 0.31 | 2.01 ± 0.19 | 11.7 ± 0.04 | 9.24 ± 0.89 |
| Protein; C (pg pg⁻¹) | 0.39 ± 0.08 | 0.44 ± 0.07 | 0.34 ± 0.07 | 0.10 ± 0.03 | 0.56 ± 0.04 | 0.70 ± 0.07 |
| NR activity | 6.99 ± 1.18 | 5.16 ± 0.05 | n.d. | n.d. | n.d. | n.d. |
| (μmol NO₂ · min⁻¹ cell⁻¹ × 10⁻¹¹) | | | | | | |
| 1/Ø (quanta fa C⁻¹) | 882 ± 91.0 | 889 ± 346 | 2728 ± 375 | 4127 ± 999 | 839 ± 91.2 | 431 ± 159 |
| 1/Ø fa C/1/Ø C | 7.09 ± 3.08 | 9.23 ± 1.29 | 7.14 ± 0.40 | 3.06 ± 0.15 | 8.42 ± 0.60 | 5.72 ± 0.82 |
| | 0.16 ± 0.07 | 0.11 ± 0.02 | 0.14 ± 0.01 | 0.33 ± 0.02 | 0.12 ± 0.01 | 0.18 ± 0.03 |

μ, growth rate;
Fv/Fm, maximum quantum yield for PSII;
Chl, chlorophyll;
a*, absorption cross-section normalized to Chl a;
C, carbon;
N, nitrogen;
FA. fatty acid;
1/Ø, quanta requirement for growth (C) and FA synthesis (Falkowski etal. 1985);
NR, assimilatory NO3− reductase activity;
n.d., Not detected.
^{a}the detailed fatty acid composition is shown in Table IIB.

TABLE IIB
Relative composition of fatty acid species (%)

| | Experimental conditions | | | | | |
|---|---|---|---|---|---|---|
| | NO₃⁻ + Mo | NO₃⁻ − Mo | NO₃⁻ − Mo + W | N-free | NH₄⁺ − Mo | NH₄⁺ − Mo + W |
| C14:0 | 5.41 ± 0.21 | 4.18 ± 1.87 | 2.84 ± 0.20 | 3.71 ± 0.29 | 4.31 ± 0.65 | 4.79 ± 0.45 |
| C16:0 | 16.3 ± 0.94 | 19.0 ± 1.81 | 22.8 ± 1.40 | 27.0 ± 2.21 | 15.3 ± 1.30 | 18.5 ± 2.89 |
| C16:1 | 24.5 ± 1.81 | 32.0 ± 0.36 | 37.0 ± 2.83 | 46.0 ± 3.76 | 28.3 ± 4.43 | 28.7 ± 5.68 |
| C16:2n4c* | 4.33 ± 0.39 | 4.30 ± 0.79 | 2.29 ± 0.15 | 1.27 ± 0.10 | 4.30 ± 0.52 | 3.69 ± 0.38 |
| C16:3n4c* | 6.37 ± 0.46 | 4.89 ± 0.60 | 2.17 ± 0.14 | 0.77 ± 0.06 | 5.73 ± 0.83 | 5.02 ± 0.91 |
| C18:1n9c | 1.14 ± 0.08 | 1.47 ± 0.16 | 0.88 ± 0.04 | 1.39 ± 0.11 | 1.28 ± 0.19 | 1.47 ± 0.15 |
| C18:2n6c | 1.60 ± 0.15 | 1.32 ± 0.56 | 1.22 ± 0.05 | 1.64 ± 0.11 | 1.06 ± 0.07 | 1.65 ± 0.41 |
| C20:5n3 | 24.8 ± 1.61 | 18.9 ± 4.11 | 17.8 ± 0.82 | 9.87 ± 0.90 | 21.8 ± 2.76 | 21.5 ± 2.31 |
| C24:0 | 3.26 ± 0.37 | 3.13 ± 0.07 | 2.90 ± 0.18 | 1.18 ± 0.11 | 3.07 ± 0.60 | 2.87 ± 0.16 |
| C22:6n3 | 3.98 ± 0.73 | 2.64 ± 0.28 | 2.51 ± 0.13 | 1.09 ± 0.09 | 3.55 ± 0.74 | 2.97 ± 0.13 |
| Total fatty acid | 2.40 ± 0.09 | 2.66 ± 0.25 | 3.99 ± 0.24 | 4.21 ± 0.33 | 4.33 ± 0.60 | 3.38 ± 0.16 |
| cell⁻¹ (pg) | | | | | | |

*identified after Alonso et al. 2000.

First we tested the effect of the exclusion of molybdenum (Mo) from the medium (NO₃⁻−Mo). The exclusion of Mo was necessary for complete inactivation of NR with sodium tungsten (W) in subsequent tests. Compared to the control condition (NO₃⁻+Mo), cells growing without Mo lost about 25% of NR activity. However, the growth rate, Fv/Fm, chlorophyll content, absorption cross-section (a*), fatty acid content and protein content (per cell and per unit carbon) and the quanta requirements for growth and fatty acid accumulation did not vary relative to the control (P≧0.05). Also in terms of daily rates of fat accumulation per cell and per unit volume we did not verify significant differences between these 2 treatments (P≧0.05; FIG. 1), besides large variation observed between samples.

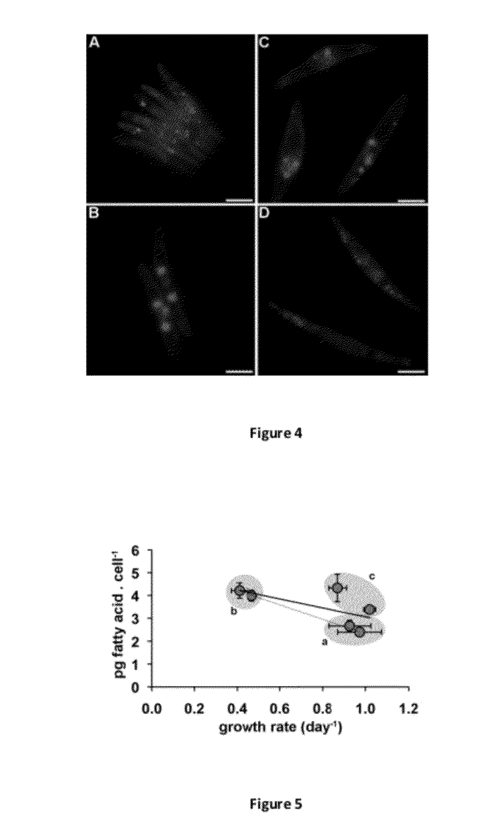

The addition of W to NO₃− growing cells (NO₃−Mo+W), led to a complete decline of NR activity to undetectable levels. In parallel, the cellular growth was severely impaired, decreasing 50%, Fv/Fm was declined 18%, the chlorophyll content declined more than 50% and the a* doubled, on average. Besides these effects, we observed microscopically than W-treated cells enlarged consistently their broad size (not measured quantitatively, see FIG. 4) and were able to retain larger amounts of total carbon and nitrogen, both being significantly superior to the control (P≦0.05), but equal C:N ratios (P≧0.05). In terms of fatty acid content cells had 40% more fat biomass than the control, however W treated cells possessed identical fat as the control per unit carbon (fatty acid: carbon) as the control (P≧0.05). In terms of protein (both per cell or unit carbon) W treated cells kept identical values to the control (P≧0.05). The overall variations relative to the control resulted in large increases in the physiological requirements for both growth and fatty acid accumulation (P≦0.05), relative to the control (2.7 and 3 times higher). In other terms both the growth and fatty acid accumulation efficiencies decreased. In N-free medium we verified stronger declines of both the growth rate, Fv/Fm of the cells relative to the control (about 57% in both parameters). The chlorophyll content also decreased more markedly and a* increased 4.5-fold. In opposition to W treated cells, in N-free medium the total pool of carbon and specially the pool of nitrogen declined significantly relative to the control (NR activity was also undetectable). Concerning the pool of fatty acid per cell, this was statistically identical between N-free medium and W treated cells. It was however noticeable that this time the amount of carbon deposited into fatty acid biomass corresponded to 45% of the total cellular carbon in N-free medium cells, whereas in the other cases, including in NO₃−Mo+W, it ranged only from 15 to 24%. These overall effects upon the cellular pools and pigment content led to more prominent increases of the quanta requirements for growth and fat accumulation, reaching 9.4 and 4.7 times higher values relative to NO₃− replete conditions. The accumulation of protein (per cell and unit carbon) was also severely impaired, declining more than 60%, in N-free medium relative to the control.

In both N-free and NO₃−Mo+W conditions, as a result of the slower specific growth rates of the cells, the cultures had the lowest daily rates of fatty acid accumulation per cell (both 20% lower than NO₃+Mo) and consequently much lower rates of fatty acid accumulation in terms of volume of culture after 3 days (both 80% lower than NO₃+Mo, FIG. 1).

With NH₄⁺ as the nitrogen source (NH₄−Mo) NR activity was also below detection. Here we did not detect significant difference in terms of growth and Fv/Fm relative to the control (P≧0.05). The increase observed in the carbon and nitrogen total pools were also significant (P≦0.05, increasing 115% and 104% respectively). Furthermore cells accumulated high levels fatty acids and protein than the control condition (45% fatty acid and 55% protein per cell), and similar amounts in terms of lipid to cells with NO₃− and W and in N-free medium (P≦0.05). We did not detect however differences relative to the control in terms of fatty acid per unit carbon (P≧0.05), but protein per unit carbon was again superior (P≦0.05). In terms of quanta requirements for growth and fatty acid accumulation, we did not detected differences compared to both the control and the NO₃−Mo condition (P≧0.05). The higher fatty acid contents per cell and elevated growth rate led however in this condition to the highest daily-rates of fatty acid accumulation per cell (FIG. 1A).

In the last growth condition where W was added to NH₄⁺ growing cultures the growth rate of the cells increased significantly (P≦0.05) more than 10% relative to the NH₄⁺ ones (keeping equivalent Fv/Fm). No differences were however noticed in terms of growth relative to the control (P≧0.05). On a cell basis the increased rate on growth led to a significant reduction of carbon and nitrogen pools as well as protein and fatty acid per cell, but the quantum requirements for growth and fatty acid accumulation were significantly lower than in all the other conditions. In terms of rate of fatty acid accumulated per unit volume, we verified significantly higher production (over 30%) compared to all the other situations (FIG. 1B).

### NR Protein Expression

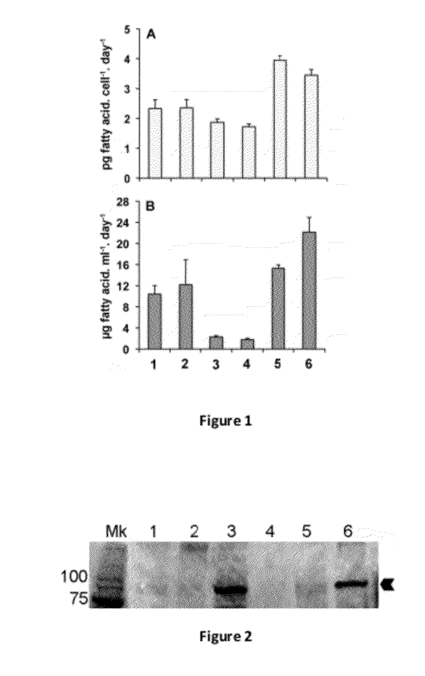

We verified by western-blot the expression of NR protein (FIG. 2). In the western-blot gel (FIG. 2) we revealed 1 NR band with approximately 100 kDa in all conditions except in cells growing in N-free medium (lane 4). NO₃− replete conditions (lanes 1 and 2) where we positively measured NR activity (Table IIA) produced very faint bands. NH4+ grown cells also produced a weak band (lane 5). The two conditions supplemented with W (lane 3, NO₃−Mo+W; lane 6, NH₄−Mo+W), revealed enhanced NR protein bands, even though there was a total absence of enzymatic activity (Table IIA). The molecular size of the identified bands (100 kDa) correlates with the estimated NR-protein size based on the NR-gene model for P. tricornutum (GM 54983, http://genome.jgi-psf.org/Phatr2/Phatr2.home.html).

### Metabolites Profiles

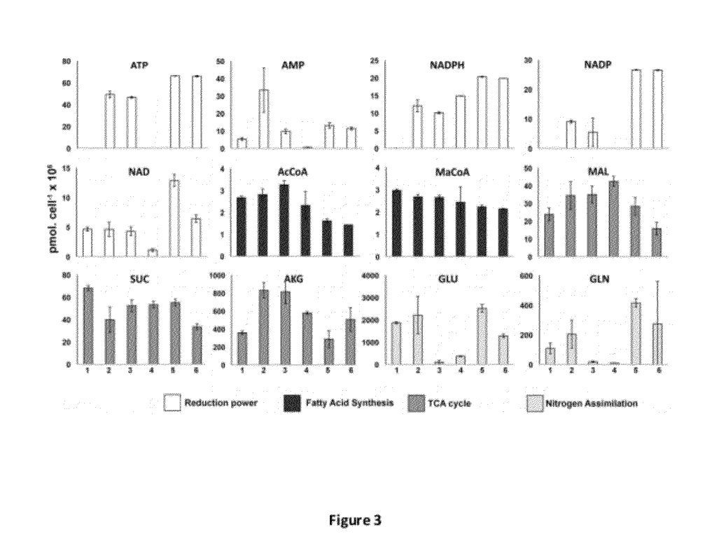

We measured many central metabolites (FIG. 3) involved in cellular energy-reduction power, TCA cycle, fatty acid synthesis, and nitrogen assimilation. The two metabolites involved in nitrogen assimilation, glutamate and glutamine, were reduced by approximately 90% in N-free and NO₃−Mo+W cells compared to the replete conditions (NO₃⁻ and NH₄⁺) as expected.

In terms of cellular energy in the form of ATP and reductant, NH₄+ (both with and without W) had the highest ATP pools (by 25%) and NO₃− with Mo and W treated cells (with NO₃−) had comparable pool sizes. We did not detect ATP for NO₃− grown cells and N-free cells. AMP was highest in NO₃−Mo. Relative to the control we verified significantly higher AMP pools in NO₃+W, but a decline in N-free medium. Both NH₄+ treatments also showed higher AMP levels than the control. NADPH was noticeably higher in both NH₄+ conditions, and similar among the other conditions. NADP+ and NAD+ were also higher in the NH₄+ conditions and similar among the others. The NADPH/NADP+ ratio was 1.3 in the NO₃⁻−Mo control and decreased to approximately 0.75 in both NH₄+ treatments, and in contrast it increased to 1.8 in NO₃−Mo+W cultures. Concerning the precursors to lipid biosynthesis, acetylCoA and malonylCoA, the acetylCoA pool was higher in NO₃+W cells, similar between control, NO₃−Mo and N-free growing cells and lower in both NH₄+ conditions (almost 50% lower than the control). For malonylCoA we did not detect an overall difference among N-free treatments and the NO₃− replete treatments, possibly due to the large variation in one N-free sample. However we did detect a significantly lower pool in both NH₄+ exposed cultures relative to the control (25% lower). We also calculated the ratios between the fatty acid synthesis related metabolites, i.e. AcCoA/MaCoA and MaCoA/total fatty acid, in order to further understand the trend of the chemical equilibrium (Table III). In NH₄⁺ growing cells the AcCoA/MaCoA ratios were significantly lower (20-25% lower than the control) than the control but no differences were detected compared with N-free medium, which presented large variations among replicates. NO₃−Mo+W had significantly higher ratios than the control (P≦0.05). Concerning MaCoA/total fatty acid, the control conditions had a significantly higher ratio, followed by the conditions with NO₃−Mo. The other conditions had 50% lower MaCoA/total fatty acid ratios, but we did not detect statistical differences between them (P≧0.05).

TABLE III
Ratios^{a} between metabolites and products of fatty acids biosynthesis (mol. mol⁻¹)

| | Experimental conditions | | | | | |
|---|---|---|---|---|---|---|
| | NO₃ + Mo | NO₃ − Mo | NO₃ − Mo + W | N-free | NH₄ − Mo | NH₄ − Mo + W |
| AcCoA/MaCoA | 0.90 ± 0.03 | 1.05 ± 0.10 | 1.23 ± 0.07 | 0.95 ± 0.39 | 0.72 ± 0.07 | 0.67 ± 0.01 |
| MaCoA/Total FA | 1.24 ± 0.04 | 1.01 ± 0.10 | 0.67 ± 0.07 | 0.57 ± 0.29 | 0.51 ± 0.14 | 0.63 ± 0.05 |

AcCoA—acetyl coA;
MaCoA—malonyl coa;
FA—total fatty acid
^{a}the standard deviation (S) of the ratios were calculated with: S_{z} = z [(Sₓ/average x)² + (S_{y}/average y)²]^{1/2}.

Among the measured TCA cycle metabolites, we verified a large variability among metabolites and samples and few clear patterns were perceptible. α-ketoglutarate presented however a lower average biomass for NH₄−Mo, which was the condition under which cells produced a larger per cell protein pool. Also, the N-free conditions showed the same or higher α-ketoglutarate pools with respect to the controls.

### Lipid Body Arrangement

The diversity of growth treatments performed in this study produced variations in the distribution and size of the lipid bodies (LB) of the P. tricornutum cells (FIG. 4). In NO₃− grown cells we could visualize small LB dispersed in the cytoplasm. The cells grown in N-free medium produced up to 3 lipid bodies densely stained with the lipophilic dye BODIPY and near to or surrounding the chloroplast (FIG. 4B). In this condition the chloroplast was considerably smaller compared to the NO₃− replete condition. With W (NO₃−Mo+W) the lipid bodies were generally smaller than in N-free cells, but it was possible to count up to 3-5 droplets also distributed close to an also reduced chloroplast. Here a broad enlargement of the cells was also noticeable (FIG. 4C). Similar to NO₃⁻ replete conditions (FIG. 4A), in NH₄⁺ growing cells the lipid bodies were very small and less densely stained, but this time they were present in very high numbers distributed along the cytoplasm (FIG. 4D). These small droplets were often densely packed in the narrower conical extremities of the cells.

NO₃−Mo and NH₄−Mo growing cells, because they were optically indistinguishable from NO3+Mo and NH₄−Mo+W respectively.

### Discussion

Three major findings are presented herein: 1) The specific inhibition of NR activity with W induced nitrogen starvation and led to augmented fatty acid per cell in a comparable fashion to cells growing in nitrogen free medium. Nitrogen deprived cultures, in N-free medium or through W treatment, yielded however low rates of fatty acid accumulation per unit volume; 2) P. tricornutum cells growing with NH₄⁺ as the nitrogen-source accumulated higher fatty acid contents per cell than with NO₃− (control) or under nitrogen starvation, while keeping elevated cellular growth rates; and 3) The addition of W to cells growing with NH₄⁺ as nitrogen-source enhanced the specific growth rate of the cells leading to higher rates of fatty acid production per volume of culture.

### NR Inactivation Induces Fatty Acid Accumulation

As previously demonstrated in other organisms (e.g. Vega 1971, Prins et al 1980, Deng 1989), the presence of W clearly eliminated NR enzymatic activity in P. tricornutum (Table HA) impeding NO₃− assimilation. This resulted in an average 40% increase in the cellular fatty acid pool (3.99±0.24 pg·cell⁻¹) relative to NO₃− replete cells (control condition) after 3 days of growth, and was comparable to the N-free condition (4.21±0.33 pg·cell⁻¹). Our initial hypothesis stating that the specific abolishment of NR activity would lead to fatty acid accumulation as in classical procedures where the medium is depleted of nitrogen was therefore positively confirmed. Nevertheless, the severe physiological effects observed in N-free medium, i.e. strong reduction of Fv/Fm, chlorophyll, C and protein contents, were clearly less pronounced with W and were also somewhat puzzling because P. tricornutum cells kept similar nitrogen, carbon and protein contents (per cell and unit carbon) as in NO₃− replete conditions and demonstrated lower (quanta) requirements for growth and fat accumulation than cells growing in N-free medium. Moreover, we verified a lower drop in the chlorophyll content and the cells maintained elevated Fv/Fm, which is often not the case for microalgae experiencing starvation (Berges et al. 1996). This ensemble of parameters questions the real extent of the limitation imposed through W-mediated NR inactivation, although glutamate and glutamine levels were equally decreased to about 10% of control levels.

Some of these observations can be further elucidated by analogy with other microalgae and higher plant systems. Indeed, in plants inactivation of NR by W appears to be generally correlated with an accumulation of NO₃− in the tissues (Heimer et al. 1969, Deng et al. 1989). In this case, NO₃− uptake appears to be therefore uncoupled from NO₃-reduction and assimilation. This process would explain the elevated total nitrogen content that we reported in P. tricornutum cells growing with NO₃− and W (35% higher than in the control condition). The accumulated NO₃− was probably stored in internal vacuoles, as it is typical in diatoms occupying large fractions of the cell volume (Eppley and Coatswoth 1968, Smayda 1970, Borowitzka et al. 1978, Dortch et al. 1984), maybe leading also to the broad enlargement of the cells observed by microscopy (FIG. 4). The milder effects on the overall cell photosynthetic capacities and carbon fixation (and biomass production) point toward a better preservation of the light harvesting machinery as well as higher efficiencies to convert light energy into fixed carbon which explain that cells harbor more carbon than cells grown in N-free medium. A possible explanation for these results is that W may not have totally abolished NR-activity or there may have been residual amounts of Mo in the medium carried along with the cell or with other nutrients in the f/2 formulation, allowing minimal nitrate incorporation and subsequent protein synthesis and functional maintenance of central processes namely photosynthesis.

However other results attest to the fact that in this growth condition the cells were indeed experiencing nitrogen starvation. First they substantially increased the fatty acid pool and they also presented very low levels of glutamine and glutamate comparable to cells in N-free medium (FIG. 3). Both amino acids are the direct products of NH₄+ assimilation after NO₃− reduction and are required for protein synthesis (Zehr and Falkowski 1988). Their low levels clearly indicate that cells were already experiencing starvation. It also follows that NH₄+ grown cells decreased their α-ketoglutarate pools, since they had the ability to uptake NH₄+ and produce glutamate and glutamine, while in N-free cells α-ketoglutarate pools built up relative to controls due to the absence of the NH₄+ substrate. Over longer periods of exposure to W, due to the lack of a nitrogen source, the state of the cells would probably tend to a scenario most closely resembling the case of cells growing in N-free medium. Indeed, Burrows et al., in a labeling study observed that over three days of NO3− deprivation, most lipid in P. tricornutum cells was synthesized de novo, indicating that the full effects of NO₃− deprivation were not yet seen over that time.

Another curious outcome of the addition of W to NO₃− growing cells was the over-production of NR-protein (despite the complete absence of activity) detected as a dense protein band by western-blot, which contrasted with cells in N-free medium where no NR band was detected (FIG. 2, lane 3). Why did cells overproduce NR-protein with W and not in N-free medium? Generally the presence of NO₃− is required for NR gene expression and protein synthesis (Campbell 1999, Berges 1995, Poulsen et al. 2005), which was the case in the W treatment. This fact further supports the existence of NO₃-up-take to the cytoplasm as we discussed above. The overproduction might however seem excessive, nevertheless tobacco plants and the green microalgae Chlamydomonas treated with W (and without NO₃− reduction capacity) also overexpress the NR-gene and produce high amounts of a tungsto-NR analogue unable to reduce nitrate but possessing NADH-cytochrome c reductase activity (partial activity independent of the Mo-cofactor) (Notton and Hewitt 1971, Deng et al. 1989, Vega et al. 1971). In our study the immunodetected NR-protein band probably consisted of the same tungsto-NR analogue being produced by P. tricornutum cells in the presence of W. A plausible explanation for this phenomenon could be that organisms able to sense and probably uptake NO₃− up-regulate the NR-gene and NR-protein synthesis to compensate for the ramping nitrogen limitation due to the inability to reduce NO₃−. However in our study when P. tricornutum cells were grown with NH₄+ together with W, they also over-produced NR-protein (FIG. 2, lane 6). This is a striking result because NH₄+ generally represses NR-gene expression and protein synthesis (Lomas and Gilbert 1999, Parker and Armbrust 2005), although we noticed a NR band in the western-blot gel (FIG. 2, lane 5). This would suggest that W alone induced the over-production of NR-protein and possibly NR-gene expression. The overproduction of NR-protein seen in NO₃− growing cells treated with W can therefore result from both conditions: an up-regulation of NR to compensate for the inability to reduce nitrate, but also from an inductive effect of W.

In addition to the extent of physiological effects of W, our data clearly demonstrated that solely by controlling the levels of NR activity it is possible to boost fatty acid accumulation in P. tricornutum. Along with this observation, it should be emphasized that even smaller reductions of NR activity can impact fatty acid accumulation and promote higher fatty acid accumulation per culture volume. This was demonstrated in Mo-free medium where NR activity was reduced 25% and the average fatty acid per cell and per unit volume increased 10% and 15% respectively. We observed that even in the theoretical absence of Mo, NR kept activity. This has been observed also in other microalgae (Vega et al. 1971) and could be related to a highly efficient mechanism for Mo recycling or to the presence of specialized protein carriers as it has been evidenced in plants and Chlamydomonas (Witte et al. 1998, Mendel 2005).

Our results indicate that manipulation of NR activity can be effective for enhancing lipid accumulation in P. tricornutum (or other microalgae). For such applications our results could be further improved by manipulating NR at the genetic level through genetic engineering, allowing the construction of a stable strain where NR activity would be finely modulated at specific growth stages to maximize oil yields and thus avoiding the utilization of W or other chemicals with toxic effects on the environment (Strigul et al. 2009, Strigul et al. 2010). Presently there is considerable genomic and transcriptomic information and a suite of molecular tools that have been developed to select gene targets and alter the levels of gene expression in several microalgal models, including P. tricornutum (Siaut et al. 2007, Bowler et al. 2008, Maheswari et al. 2008, De Riso et al. 2009).

Notably, any microalgal-biodiesel production system will require not only optimized oil yields per cell, but most importantly optimized rates of oil biomass production per unit volume. A real problem with strategies involving starvation to enhance lipid production is that growth rates are severely reduced, limiting the rates of biomass production and yields per volume of culture (FIG. 1). In these terms, one may wonder if strategies based on nutrient limitations are really suitable for biodiesel mass production. Probably not, unless, as it has been proposed elsewhere (Radakovits et al., 2010), cells are kept at elevated concentrations in a steady-state stage with impaired growth, budding-off lipids (or full lipid bodies) to the medium, like the secretion of lipid globules by mammalian milk glands (Murphy and Vance 1999). These lipids would then emerge to the surface of the bioreactor and be harvested.

### NH₄⁺ as Nitrogen Source Enhances Fatty Acid Accumulation

Growth limitations are less of a problem when NH₄+ is provided as nitrogen source, since microalgae keep elevated growth rates, generally comparable or superior to NO₃− grown cells (e.g. Levasseur et al. 1993). Concerning P. tricornutum it has been reported that when NH₄+ is provided in a non-buffered medium cells die after 3 days (Yongmanitchai and Ward 1991). In our study (using a different strain) however we did not verify this effect. Cells remained healthy (high Fv/Fm) and possessed similar growth rates to NO₃− grown cells (in this section we refer solely to NH₄−Mo, unless indicated).

Concerning the accumulation of fatty acid, P. tricornutum cells growing with NH4+ accumulated ≧40% fatty acid per cell than NO3− grown cells and surprisingly similar amounts to nitrogen starved cells (in N-free medium and growing with NO3− and W). This latter result was clearly unexpected since generally cells growing without nutrient limitation do not accumulate excess fatty acid biomass (Hu et al. 2008). To our knowledge only one study reported higher fatty acid contents for algae with NH₄+ as the nitrogen source relative to NO₃− (Xu et al. 2001), but no study reported similarities between the lipid content of NH₄+ growing cells and nitrogen limited cells.

In addition to the fatty acid, NH₄+ grown cells also fixed more carbon and synthesize more protein per cell than NO₃− growing cells (and the other conditions). Based on these observation one would expect that P. tricornutum cells growing with NH₄+ would show clearly higher efficiencies (1/quanta requirement) at converting light energy into biomass production. However this was not observed. NH₄+ grown cells possessed similar efficiencies to grow and accumulate fatty acids to NO₃− grown cells.

The contradiction observed between measured fatty acid content and the calculated quanta efficiencies for growth and fat accumulation, might be explained by the dependency on the growth rate of the model used to calculate the quantum yields (Falkowski et al. 1985). In fact although statistically identical, an average 10% drop on the growth rates of NH₄+ compared to the control was observed. This potential drop in growth resulted in a decrease in the calculated efficiencies and in the apparent similarities of transduction of light energy into biomass. Other evidence from our study, points however to the fact that NH₄+ grown cells favored fat accumulation relative to NO₃− grown cells.

This can be visualized by plotting the fatty acid biomass per cell (pg) as a function of the growth rate of all conditions (FIG. 5). Growth rate reduction leads to a proportional increment of fatty acid per cell. This can be clearly observed between NO₃− and starved cells (groups a and b in the FIG. 5). Higher fat accumulation in slow growing cells is expected since cells and internal pools divide slower, therefore resulting in intracellular accumulation. However this trend is not applicable to NH₄+ growing cells, which appeared above the trendline accumulating more fatty acid than what would be predicted based solely on their growth rates. This suggests that beyond the slight reduction of growth relative to NO₃− growing cells, other processes were fomenting fat accumulation when NH₄+ was provided as the nitrogen source.

Further evidence for a mechanism fomenting fatty acid accumulation can be further pointed out by calculating the ratio between consecutive metabolites involved specifically in the lipid biosynthetic pathway and the final products (fatty acid) across all experimental conditions (Table III). Overall NH₄+ conditions presented lower ratios of AcCoA/MaCoA and MaCoA/fatty acid than the control and identical or lower ratios than nitrogen starved conditions. This indicates the existence of a lower disequilibrium between reagents and products in NH₄+ conditions; possibly due to the existence of a higher flux of carbon through this pathway via faster conversion of substrate to product. This would explain why cells in these conditions accumulated higher amounts of fat.

When microalgae uptake NH₄+ instead of NO₃− (Eppley et al. 1969, Syrett 1981) they bypass the consecutive reductions of NO₃− to NO₂— and from NO₂— to NH₄+ by NR and nitrite reductase (NiR) respectively (Campbell 1999, Levasseur 1993). By doing so, cells reduce the considerable energetic costs (reductant, NAD(P)H) associated with NO₃− and NO₂ reduction (Falkowski 1975, Turpin and Bruce 1990, Levasseur et al. 1993). Under identical irradiance (namely high irradiances, like we used in our study, i.e. 300 mmol quanta·m⁻²·s⁻¹) and CO₂ availability, the higher levels of free energy made available by direct assimilation of NH4+ can be diverted to some extent for growth but should be also managed to avoid cellular damage. Several sinks for excess energy have been described in microalgae, namely the NO₃− up-take and reduction pathways detailed above (Lomas and Gilbert 1999L&O), photorespiration (Wingler et al. 2000, Parker et al. 2004), the xanthophyll cycle (Ruban et al. 2004), cyclic electron flow around photosystem II (Lavaud et al. 2002, Feikema et al. 2006), and chlororespiration (Dijkman and Kroon 2002). Further downstream, the de novo synthesis and accumulation of macromolecular pools, especially TAG, might be used as well as a sink for energy dissipation. For example the formation of a C18 fatty acid consumes approximately 24 NADH derived from the photosynthetic electron transport chain. This is twice that required for synthesis of carbohydrate or protein molecules of the same mass, and thus relaxes the over-reduced electron transport chain under excess energy avoiding the overproduction of hazardous reactive oxygen species (Hu et al. 2008). Based on this assumption we therefore hypothesize that P. tricornutum cells produced more fatty acid contents in NH₄+ growing cells as a mechanism to manage excess light energy and avoid photo-damage. Based on this interpretation it would be predictable that higher light conditions would promote even higher fatty acid accumulation in NH₄+ growing cells

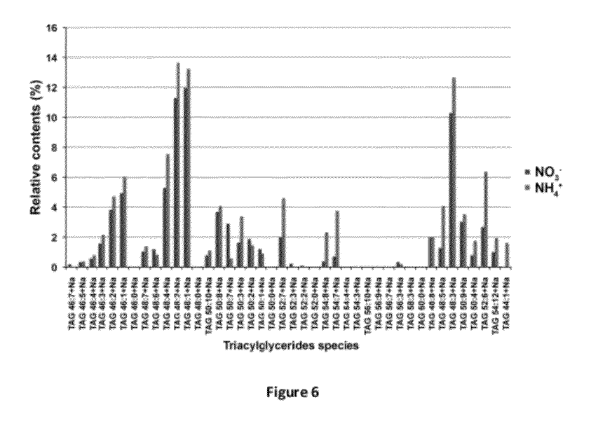

We investigated what type(s) of lipid(s) are increased with NH₄+. Preliminary analyses made by liquid-chromatography mass-spectrometry indicate that all TAG species of P. tricornutum NH₄+ grown cells increase relative to NO₃− grown cells (See FIG. 6) further supporting the idea that the cells utilize these storage materials as a sink for excess of energy.

We also noted the concomitant increment on protein content with NH₄+.

Tungsten Enhances Growth Rate on NH₄+ grown cells

One of the most striking results of our study was that W boosted the specific growth rate of cells grown with NH₄+ as the nitrogen source (Table II). In fact with W P. tricornutum cells grew 10% faster than cells growing solely with NH₄+ (NH₄−Mo) equaling the average growth observed with NO₃−. This increased rate of cell division led to a reduction of the cellular fatty acid pool relative to cells on NH₄+ solely (pg·cell⁻¹), however it promoted the highest efficiencies of fatty acid production and consequently the rates of production of fatty acid per volume of culture, rendering the effect of W very attractive in terms oil production.

Studies on the effect of W upon organisms are invariably related to the inactivation of NR (as discussed previously). There are however some scarce reports that indicate that W (as sodium tungsten as in our study) in small concentration (≦400 μM) stimulated growth of a freshwater microalgae, Selenastrum capricornutum (Strigul et al. 2009). Another reference by the same first author indicated that W was used as a leaf fertilizer for viticulture in Russia (Strigul 2009). However, to our knowledge, there is no information on the mechanism by which W promotes growth.

Is there a particular enzyme that requires W (absent in the utilized artificial medium), therefore limiting P. tricornutum cell growth? This is probably not the case, because eukaryotes do not have W-dependent enzymes according to the current knowledge (Schwarz et al. 2007). Moreover, we did parallel assays using seawater-based medium and we observed the same growth enhancement in P. tricornutum (data not shown). Alternatively, did Mo replacement by W in a particular (or in some) enzyme(s), promote the over-induction of its activity that would subsequently promote growth? It is known that W can substitute for Mo in all four eukaryotic Mo-enzymes (sulfite oxidase, nitrate reductase, xanthine oxidase, aldehyde oxidase) studied so far (Mendel 2005, Schwarz et al. 2007). Finally, does W have other types of effects upon cells physiology? We verified in the western-blot gel (FIG. 2) that in the presence of NO₃− or NH₄+ plus W, P. tricornutum cells over-produced an inactive NR-protein (lane 6, FIG. 2). This was totally unexpected especially in the case of NH₄+, because NH₄+ is an inhibitor of NR-gene expression and NR-enzyme synthesis (Lomas and Gilbert 1999, Parker and Armbrust 2005). As we discussed in a previous section, W could therefore function as an inducer for NR-gene expression.

Searching for the effects of W is fundamentally relevant to further understand its mode of action and the effects upon cellular growth, but from a biotechnological point of view, the discovery of key regulators controlling cell growth would allow further genetic manipulations of cell growth, which could have several applications including the promotion of the rates of oil production.

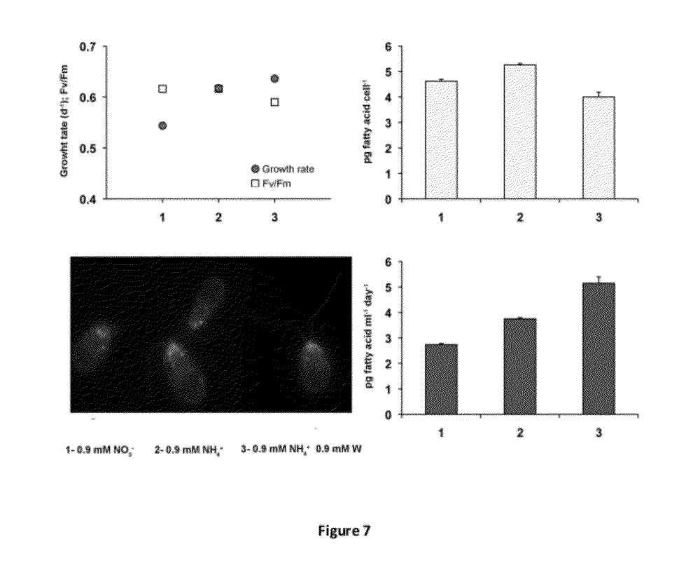

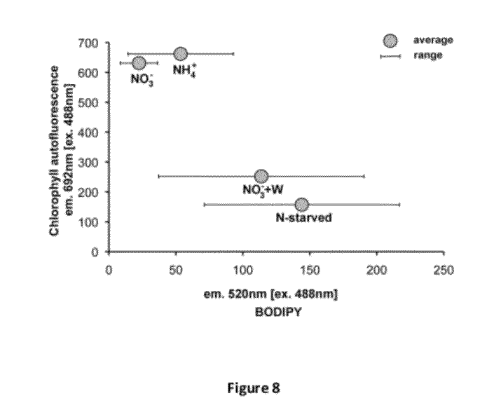

A last question emerging for this study is whether the combined effect of NH₄+ and W is specific to P. tricornutum or whether it can be expanded to other microalgae. To address this question we performed the same experiment with the green microalgae Dunaliella terciolecta. Similarly to P. tricornutum, D. terciolecta cells growing with NH₄+ accumulated 13% more total fatty acid than with NO₃−. When W was provided in conjunction with NH₄+ Dunaliella cells also increased the average growth rate and produced after 3 days, 88% more fatty acid per unit volume than nitrate-grown cultures. See FIG. 7. Based on these data, it appears that the effect of W on growth of cells with NH₄+ as the nitrogen source extends to species in other classes of microalgae

### Lipid Body Size and Distribution

Lipid bodies (LBs) are ubiquitous in miroalgal cytoplasm including microalgae (Murphy 2001) or chloroplasts (Ben-Amotz et al. 1989), being generally composed of a monolayer of amphipatic lipids encircling a hydrophobic core of neutral lipids, namely TAGs. A curious observation of our work was that cells under starvation (both in N-free medium and with W) produced few but large LB surrounding the chloroplast, whereas cells growing with NH₄+ as the nitrogen source and possessing higher effective total fatty acid pools measured by gas-chromatography produced instead large amounts of minute LBs scattered in the cytoplasm often filling the conical extremities of the cells (FIG. 4). Why these differences? This question opens a wider spectrum of issues questioning the underlying mechanism regulating LBs size and distribution under starvation or different nutrient regimes and the overall functionality of LBs. Indeed in addition to a storage function, LBs (and overall TAG synthesis and accumulation) may play more active and central roles in stress response, for instance in buffering excess energy (discussed previously). Furthermore TAG synthesis is usually coordinated with carotenoid (b-carotene, lutein, astaxanthin) synthesis, which are esterified and also sequestered in LBs.

In fact nitrogen starved cells also increase their carotenoid contents and peripheral distribution of these carotenoids serves as “sunscreen” to prevent or reduce excess light striking the chloroplast under stress (Rabbani et al. 1998, Hu et al. 2008). It is possible therefore that the close association of LBs to the reduced and barely pigmented chloroplasts in starved P. tricornutum cells might increment photoprotection, whereas in non-limited cells, possessing large and well pigmented plasts, carotenoid mediated photoprotection is less required and LBs appear scattered in the cytoplasm. On the other hand, the large size of the LBs under starvation may be related to the lack of membrane lipids (phospholipids), which are known to decrease markedly under starvation in P. tricornutum and other diatoms (Parrish and Wangersky 1987, Lynn et al. 2000) being a limiting factor for LBs formation and abundance. In fact in Drosophila when a rate-limiting enzyme for phospholipid biosynthesis is inactivated, lipid droplet increased in size presumably because larger droplets have a lower surface area-to-volume ratio and require less phospholipids to form the monolayer around the LB (Guo et al. 2008 Nature). Cells growing in non-limited conditions are not limited in membrane lipid synthesis and therefore can produce smaller LB that might be easier to manage intracellularly.

Several procedures to rapidly screen microalgal oil contents have been developed. In most of them the cells are stained with lipophilic dyes (Nile Red and the fluorescent dye BODIPY) and then analyzed by spectrophotometry or flow cytometry to measure the relative lipid contents (e.g. de la Jara et al. 2003, Chen et al. 2008, Yu et al. 2009, Cooper et al. 2010). Previous attempts have been made in our laboratory to use flow cytometry to screen microalgae stained with BODIPY. In the frame of the results presented in this study it was curious that cells growing in NH₄+ and possessing the highest fatty acid contents measured by gas chromatography yielded similar fluorescent intensities as nitrate-growing cells that had much lower fatty acid contents and much lower fluorescent intensities than P. tricornutum cells growing in N-free medium or with NO₃− and W. See FIG. 8. This fact may be the result of the scattered distribution, lower size and maybe different optical properties of the LBs of NH₄+ growing cells. However, these observations highlight inaccuracy of screening procedures based on colorimetric or fluorescent dyes, and also highlight the importance for maintaining classical chromatography methods, that are evidently slower, but provide accurate and quantitative measurements of organism lipid contents, independently of the growth condition or phenotypic variations. Nevertheless the visual lipid staining results provide valuable physiological data on LB size and number that is missed with chromatography alone.

In summary, we have shown that inhibition of nitrate reductase enzyme activity with tungsten induces nitrogen starvation and consequently stimulates lipid accumulation. We have also shown that ammonium can serve as the sole nitrogen source. Using tungsten in combination with ammonium resulted in higher growth rates and lipid accumulation which was not species specific. The product of altering these two variables lead to 87% higher rates of lipid production per unit biomass than in controlled conditions and was significantly higher than that observed in nitrogen starved cells. Using this inexpensive approach, a doubling of lipid production is achievable from algal cells.

### REFERENCES

Bennette N B, Eng J & Dismukes G C (2010) Targeted metabolite quantitation in the hydrogenic cyanobacterium Synechococcus sp. PCC 7002 by Liquid Chromatography—Mass Spectrometry; deciphering anoxic energy metabolism towards enhanced biofuel production.
Dubois M, Gilles K A, Hamilton J K, Rebers P A, and Smith F (1956) Colorimetric method for determination of sugars and related substances. Anal. Chem., 28 (3): 350-356.
Gorbunov, M. Y. & Falkowski, P. G. (2005) Fluorescence induction and relaxation (FIRE) technique and instrumentation for monitoring photosynthetic processes and primary production in aquatic ecosystems. Photosyntheis: Fundamental aspects to global perspectives, A. van der Est and D. Bruce Eds. International Society of Photosynthesis.
Deng M, Moureaux T, Caboche M. 1989. Tungstate, a molybdate analog inactivating NO3− reductase, deregulates the expression of the NO3− reductase structural gene. Plant Physiology 91, 304-309.
Thompson (1989) Membrane acclimation by unicellular organisms in response to temperature change JOURNAL OF BIOENERGETICS AND BIOMEMBRANES, Volume 21, Number 1, 43-60.
Mock and Kroon 2002 Photosynthetic energy conversion under extreme conditions—II: the significance of lipids under light limited growth in Antarctic sea ice diatoms. Phytochemistry
NITROGEN LIMITATION IN ISOCHRYSIS GALBANA (HAPTOPHYCEAE). II. RELATIVE ABUNDANCE OF CHLOROPLAST PROTEINS Paul G. Falkowskil,*, Assaf Sukenik2, Ronny Herzig3 Volume 25, Issue 3, pages 471-478, September 1989

While certain of the preferred embodiments of the present invention have been described and specifically exemplified above, it is not intended that the invention be limited to such embodiments. Various modifications may be made thereto without departing from the scope of the present invention, as set forth in the following claims.

What is claimed is:

1. A method for increasing lipid production and growth rate in marine alga comprising:
a) culturing microalgae in the presence of NH₄⁺ as the sole nitrogen source;
b) exposing said culture to an effective amount of tungsten, said tungsten causing inactivation of nitrate reductase; the combination of steps a) and b) resulting in elevated fatty acid accumulation and enhanced growth rate when compared to microalgal cells grown in NO₃− replete and nitrogen free medium in the absence of tungsten and NH₄⁺.

2. The method of claim 1, wherein said lipids are used for biofuel production.

3. The method of claim 1, wherein said microalgae is P. tricornutum.

4. The method of claim 1, wherein said microalgae is D. terciolecta.

6. The method of claim 1, wherein said microalgae are grown under high light conditions.

7. The method of claim 6, wherein said microalgae are maintained at an irradiance of approximately 950 μE.
